(19) United States
(12) Patent Application Publication
(10) Pub. No.: US 2008/0275107 A1
(43) Pub. Date: Nov. 6, 2008

(54) TOPICAL FORMULATIONS AND METHODS FOR ELIMINATION AND PROPHYLAXIS OF SUSCEPTIBLE AND TREATMENT RESISTANT STRAINS OF HEAD LICE WITH MULTIPLE MODES OF ACTION

(76) Inventor: Nicholas Spring, Jenkintown, PA (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 12/115,291

(22) Filed: May 5, 2008

Related U.S. Application Data
(60) Provisional application No. 60/916,107, filed on May 4, 2007.
(60) Provisional application No. 60/976,259, filed on Sep. 28, 2007.

(51) Int. Cl. A61K 31/351; A61P 33/00

(52) U.S. Cl. 514/450

(57) ABSTRACT

Topical formulations for prophylaxis or elimination of susceptible and treatment resistant head lice wherein the formulation is an aqueous dispersion of an oil phase containing an effective amount of an active ingredient component combining at least one compound that binds to glutamate-gated chloride ion channels and at least one compound that binds to gamma-amino butyric acid (GABA) receptors, dissolved in at least one water-soluble or water-miscible surface active agent with one or more suspending agents and one or more non-ionic surfactants. Also disclosed are methods of using the topical formulations to treat or prevent an infestation of either a susceptible or treatment resistant strain of head lice in a human patient.

### CROSS-REFERENCE TO RELATED APPLICATION

The present application claims priority benefit under 35 U.S.C. §119(e) to Provisional U.S. Patent Application Ser. No. 60/916,107 filed May 4, 2007 and Provisional U.S. Patent Application Ser. No. 60/976,259 filed Sep. 28, 2007, the disclosures of both of which are incorporated by reference.

### FIELD OF THE INVENTION

The present invention relates to topical formulations containing a combination of at least one compound that binds to glutamate-gated chloride ion channels and at least one compound that binds to gamma-amino butyric acid (GABA) receptors for use in the prophylactic and therapeutic remedy of a treatment resistant and/or susceptible head lice infestation in humans. The present invention also includes methods for treating an infestation of head lice that comprise administering one or multiple doses of the disclosed topical formulations to a human patient, wherein the infestation being treated represents a susceptible and/or treatment resistant strain of head lice.

### BACKGROUND OF THE INVENTION

Head lice, Pediculus humanus capitis, are parasitic insects that mainly live and feed in the scalp and neck hairs of human hosts. A typical infested scalp of a patient with head lice houses about 20 female lice, which are prolific egg layers over the course of their 30-day life cycle. These eggs are cemented to the hair shaft and are commonly called nits. Head lice have a similar appearance to wingless ants. Like all insects, the parasite has a body that is constructed of a hard chitinous exoskeleton. The egg case that surrounds the nits is of similar construction and is glued to the hair shaft via a cement that is similar in characteristics to the hair keratin itself. Infections, irritation and lesions to the scalp are common side effects of head lice infestation. Head lice infestation is an oft occurring problem in the United States and is easily spread from direct head-to-head contact with an infected person's hair and common usage of combs and clothing.

There are between 6 to 12 million annual head lice infestations in the United States. Grade school children suffer most from head lice infestations, with one result being an accumulation of absences from school, where lice outbreaks are believed to account for 12 million to 24 million missed days a year. A majority of public schools have instituted a no lice or a no lice and no-nit policy, forcing absenteeism on children and maybe forcing working parents to stay at home to look after them. For a review, see Burkhart and Burkhart (2006, Expert Opin. Drug Saf. 5(1):169-179).

Treatment for eliminating head lice traditionally included home remedies such as smearing mayonnaise, olive oil, a hair pomade, or some heavily viscous material about an infested scalp coupled with rigorous combing of the hair and meticulous removal of adult lice, nymphs, and nits. Though these home remedies do not kill head lice, the prevailing thought is that the viscosity of the material makes it hard for head lice to roam about the scalp, making for easy removal. Such home remedies are usually ineffective at controlling head lice due to the ability of the lice to revive rapidly once these materials are removed.

More effective treatments for eliminating head lice involve massaging the infested scalp with over-the-counter (OTC) topical creams containing active insecticides. Because of their potential toxicity to the human host, the use of these topical formulas are regulated by the FDA. Over-the-counter insecticides typically have pyrethrins or permethrin as active ingredients.

Pyrethrins are any one of six naturally occurring insecticides extracted from the chrysanthemum flower. Along with its synthetic derivative permethrin, these molecules act on susceptible head lice by increasing sodium levels in the nervous system of the lice. The increased sodium levels cause membrane depolarization in the nervous system of the head lice, which eventually leads to spastic paralysis and death.

When first introduced, both pyrethrin and permethrin were highly effective at eliminating susceptible lice. In the late 1980's, various formulations of both active ingredients had a high efficacy for eliminating adult head lice and their nits. However, recent reports indicate that treatment resistant strains of head lice have evolved for Nix®, having 1% permethrin as an active ingredient, and various Rid® products, having approximately 0.33% pyrethrin as active ingredients. It comes as no surprise that strains of treatment resistant head lice have been identified in both the United States and Europe due to the similar killing pathway for both insecticides.

Prescription products are also currently available and contain either lindane or malathion as the active ingredient. The insecticides specifically target the nervous system of the head lice. The chlorinated hydrocarbon, lindane, is formulated as a topical shampoo and is prescribed to treat head lice infestations. Lindane eliminates head lice by effectively slowing the insect's central nervous system causing paralysis and eventual death.

The reports of continued effectiveness of lindane for eliminating head lice is inconsistent at best. Reports on the efficacy of lindane for treating head lice have been listed from 17% in the U.S. to as high as 61% and 93% in other parts of the world. However, treating head lice with lindane poses problems for the human host. Side effects include toxicity to the central nervous system, convulsions, seizures; and it may be a carcinogen. Lindane has also been reported to have a slow killing time, and has poor ovicidal capacities.

Malathion is also available as a prescription insecticide to treat head lice infestations (e.g., OVIDE® an alcohol based lotion containing 0.5% malathion, terpeneol, dipentene and pine needle oil in 78% isopropyl alcohol). It is an organophosphate that causes spastic paralysis and death in head lice.

As noted above, recently there has been an increase in strains of head lice which show resistance to available OTC and prescription treatments for head lice infestation. These parasites have adapted to chemical treatments using pyrethrins, permethrins, lindane and malathion. In the US permethrin resistant lice have been found in Massachusetts, Idaho, Texas, California and Florida. Single and dual resistance to pyrethrins, permethrins and malathion has been broadly reported in Great Britain (see Downs et al., 1999, Br. J. Dermatol. 141:508-511).

Strains of head lice have been identified worldwide which are resistant to all currently available topical treatments. Possible neural damage to the human host prevents raising the insecticide levels above the current threshold in an attempt to combat these newer treatment resistant head lice. One possible way to address this alarming increase in treatment resistant head lice would be to develop and introduce a topical pediculicide formulation which is (i) safe; (ii) effective against head lice infestation from both susceptible and treatment resistant strains; (iii) is convenient for patient use; and, (iv) has not previously been marketed for such an indication. Such a strategy is disclosed herein by utilizing an anthelmintic agents derived from avermectin.

Avermectin is a natural fermentation product derived from the soil bacterium Streptomyces avermitilis. Avermectin naturally occurs as abamectin, a mixture of avermectin isomers containing >80% avermectin B₁ₐ and <20% avermectin B_{1b}, see FIGS. 1A and 1B, respectively. Other semi-synthetic forms of avermectin and mutated forms of Streptomyces avermitilis containing avermectin, for example doramectin, ivermectin, selamectin, and eprinomectin have found medicinal uses as well.

Ivermectin is the synthetic dihydro form of avermectin and is an effective insecticide. Cully et al., J. Biol. Chem., 271(33), 20187-20191 (1996) disclose these compounds to selectively bind with high affinity to glutamate-gated chloride ion channels, thus blocking chemical transmission across nerve synapses that utilize glutamate. This blockage, which occurs in invertebrate nerve and muscle cells, leads to an increase in the permeability of the cell membrane to chloride ions with hyperpolarization of the nerve or muscle cell, resulting in paralysis and death of the parasite.

The selectivity of ivermectin is attributable to the fact that some mammals do not have glutamate gated chloride channels and that the compound has a low affinity for mammalian ligand-gated chloride channels. In addition, ivermectin does not readily cross the blood brain barrier in humans. Thus, ivermectin has a documented history of highly safe and efficacious use in humans and animals. For example, over 400 million doses of orally formulated ivermectin have been used for controlling river blindness since 1986.

Ivermectin is commercially available as STROMECTOL® for eradication of Strongyloides stercoralis, which causes strongyloidiasis, and Onchocerca volvulus, which causes onchocerciasis. Ivermectin is also available as MECTIZAN® for eradication of Onchocerca volvulus and Wuchereria bancrofti. Ivermectin is usually available as a mixture containing at least 90% 5-O-demethyl-22,23-dihydroavermectin Ala and less than 10% 5-O-demethyl-25-de(1-methylpropyl)-22,23-dihyro-25-(1-methylethyl avermectin Ala, generally referred to as 22,23-dihydroavermectin B₁ₐ and B_{1b}, or H₂B₁ₐ and H₂ B_{1b}, respectively, see FIGS. 2A and 2B respectively.

Glaziou et al. (1994, Trop. Med. Parasitol. 45: 253-254) disclose treating humans with a single oral 200 ug/kg dose (n=26). Oral ivermectin was effective at this concentration against head lice. A second dose was suggested for prophylaxis, but not as part of the initial therapeutic regime.

Youssef et al. (1995, Amer. J. Trop. Med. Hyg. 53(6):652-653) describe a method of topical application of ivermectin to treat head lice.

Dunne et al. (1991, Trans. R. Soc. Trop. Med. Hyg. 85: 550-551) disclose results from a study wherein ivermectin was administered as a single oral dose of 100-200 ug/kg to treat head lice infestation. Positive, but not absolute, results where reported for this oral dosing regime.

U.S. Pat. No. 4,199,569, issued Apr. 23, 1980, discloses ivermectin, which as noted above is a semi-synthetic, anthelmintic agent derived from the avermectins, a class of highly active broad-spectrum anti-parasitic agents isolated from the fermentation products of Streptomyces avermitilis.

U.S. Pat. No. 6,103,248, issued to Burkhart and Burkhart, disclose a topical formulation for the treatment of head lice which includes a killing agent, and a lipophilic carrier having a viscosity within a range of from about 10,000 centipoise to about 85,000 centipoise at 21° C.

U.S. Pat. No. 6,524,602, also issued to Burkhart and Burkhart, disclose a topical formulation which includes a parasiticide and N,O-carboxymethyl-chitosan polymer, and a vehicle for the parasiticide and polymer.

U.S. Pat. No. 7,064,108, issued to Guzzo, et al., discloses an ivermectin-based topical gel composition comprising a pharmaceutically acceptable alcohol (30-40%; e.g., propylene glycol), a pharmaceutically acceptable glycol (30-40%; e.g., ethyl alcohol), and a pharmaceutically acceptable carrier. Ivermectin is contemplated at a w/v basis from 0.005 to 1.0%. Additional additives may include d-limonene, a non-ionic surfactant and a pharmaceutically acceptable viscosifying agent (e.g., hydroxypropylcellulose).

U.S. Pat. No. 5,952,372, issued to William McDaniel, discloses methods of treating rosacea in humans involving orally-administered or topically-applied ivermectin. The topical aspect of the invention suggests a topical formulation with about 2% ivermectin.

U.S. Pat. Nos. 6,399,652; 6,399,651 and 6,319,945, issued to L. Dean Parks, disclose methods of treating skin disorders via application of topical formulations containing ivermectin to treat acne vulgaris (the '652 patent), a variety of dermatoses (e.g., transient acantholytic dermatitis, acne miliaris necrotica, acne varioliformis, perioral dermatitis, and acneiform eruptions; the '651 patent) and seborrheic dermatitis (the '945 patent).

U.S. Pat. No. 6,262,031, issued to Larouche, et al. discloses an oral formulation of ivermectin to treat a head lice infestation.

In addition to avermectins, spinosyns are also used in the present invention as an active ingredient to eliminate both treatment resistant and susceptible strains of human lice infestations. Spinosyns are insecticides used to treat various agricultural insect infestations. Spinosyns are fermentation by products of the bacterium Saccharopolyspora spinosa. Spinosyn has many molecular variations, for example spinosyn A (known as A83543A) and spinosyn D (known as A83543D) as illustrated in FIG. 3.

Spinosyn A and spinosyn D are sold as a mixture in commercial pesticides called Spinosad, where Spinosad is sold by DowAgros'Conserve® SC, Entrust®, and the pesticide Monterey Garden Insect Spray® as manufactured for Lawn and Garden Products Inc. Spinosyns have been shown to selectively bind with high affinity to gamma-aminobutyric acid (GABA) receptors, thus blocking chemical transmission across nerve synapses that use GABA, which causes a lethal excitation of an insect's nervous system. The World Health Organization lists spinosyns as unlikely to be hazardous to humans.

U.S. Pat. No. 6,727,228, issued to Janssen et al., includes a composition for killing adult lice and their ova comprising a mixture of spinosyn A and spinosyn D and pharmaceutically acceptable carriers.

U.S. Pat. No. 6,342,482, issued to Synder, includes shampoo and conditioner formulations for controlling lice infestations in humans.

U.S. Pat. No. 7,030,095, issued to Janssen et al., includes method of controlling adults and ova of the species Anoplura with a topical composition including mixtures of spinosyn A and spinosyn D and pharmaceutically acceptable vehicles.

U.S. Pat. No. 6,063,771, issued to Snyder, includes a pediculicidal shampoo comprising spinosyn, a synthetic surfactant, an amide, and water.

U.S. Pat. No. 6,927,210, issued to Thompson et al., includes a stable ectoparasiticidal aqueous suspension of spinosyn for administration to animals.

It is evident that the OTC or prescription products presently available to the public for treatment or prevention of head lice each have their own significant drawbacks. Over-the-counter products such as pyrethrin and permethrin are presently compromised due to emerging strains of treatment resistant head lice. On the other hand, prescription products such as lindane and malathion carry recognized health risks and are also losing effectiveness due to the emergence of treatment resistant lice.

To this end, there remains a need for a patient friendly product that is safe and effective in treating susceptible and/or treatment resistant head lice.

### SUMMARY OF THE INVENTION

The present invention addresses and meets this need and is based on the realization that ectoparasite populations would rarely develop resistance simultaneously to agents that selectively bind to glutamate-gated chloride ion channels and agents that selectively bind to GABA receptors. This discovery can be applied to the treatment of infestations of resistant lice strains, as well as to the treatment of susceptible lice strains in order to prevent the emergence of resistance.

The present invention also incorporates the discovery that both modes of action are also effective on louse nymphs, so that formulations according to the present invention can also be applied effectively to susceptible and resistant lice as they are developing in or emerging from the nit.

Accordingly, the present invention provides pediculicidal topical formulations combining compounds that selectively bind to glutamate-gated chloride ion channels with compounds that selectively bind to GABA receptors, as exemplified by avermectin/spinosyn based topical formulations and more specifically by ivermectin/spino-sad based topical formulations. The present invention also discloses methods of use, which are safe, that will appeal to the patient for ease of use. The present invention is shown to be effective against multiple strains of either susceptible and treatment resistant head lice.

The present invention relates to a topical formulation for use in the prophylaxis or elimination of head lice (Pediculus humanus capitis) on a human host, wherein the formulation is an aqueous dispersion of an oil phase containing a pharmaceutically effective amount of an active ingredient component combining at least one compound that selectively binds to glutamate-gated chloride ion channels and at least one compound that selectively binds to GABA receptors, wherein the active ingredient component is dissolved in at least one water-soluble or water-miscible surface active agent with one or more suspending agents and one or more non-ionic surfactants.

The present invention also relates to a topical formulation for use in the prophylaxis or elimination of head lice (Pediculus humanus capitis) on a human host, wherein the formulation is an aqueous dispersion of an oil phase containing a pharmaceutically effective amount of an active ingredient component combining at least one compound that selectively binds to glutamate-gated chloride ion channels and at least one compound that selectively binds to GABA receptors, wherein the active ingredient component is dissolved in at least one water-soluble or water-miscible surface-active agent with one or more suspending agents and one or more non-ionic surfactants.

According to one embodiment of the present invention, both the glutamate-gated chloride ion channel-binding compounds and the GABA receptor-binding compounds are macrocyclic lactones, or macrolides. More specifically the active ingredient component contains at least one glutamate-gated chloride-ion channel-binding compound that is an avermectin, and even more specifically is an ivermectin, or a physiologically acceptable derivative or salt thereof, and at least one GABA receptor-binding compound that is a spinosyn, or a physiologically acceptable derivative or salt thereof, and even more specifically is spinosad.

In additional embodiments of the present invention, the topical formulations may further contain additional beneficial agents; non-limiting examples being humectants, preservatives, nutrients and vitamins. The present invention also includes embodiments without pharmaceutically accepted glycols.

The present invention further relates to a method of treating a head lice infestation in a human patient which comprises topically administering to the patient a formulation containing a pharmaceutically effective amount of an active ingredient component combining at least one compound that selectively binds to glutamate-gated chloride ion channels and at least one compound that selectively binds to GABA receptors via one or multiple doses; the strain of head lice being either a susceptible strain, a treatment resistant strain, or any combination of either or both types of strains within a specific human infestation.

The present invention also relates to a method of treating a head lice infestation in a human patient which comprises topically administering to the patient a glutamate-gated chloride ion channel-binding and GABA receptor-binding formulation as disclosed herein by one dose or multiple doses; the strain of head lice being either a susceptible strain or a treatment resistant strain or any combination of either or both types of strains within a specific human infestation.

Another aspect of the present invention relates to the use, in manufacture of a topical medicament for treating or preventing a head lice infestation from a treatment resistant strain in a human patient, of an active ingredient component combining at least one compound that selectively binds to glutamate-gated chloride ion channels and at least one compound that selectively binds to GABA receptors.

Another aspect of the present invention relates to the use, in the manufacture of a medicament for treating or preventing a head lice infestation from a susceptible or treatment resistant strain in a human patient, of a topical formulation according to the present invention.

The present invention further relates to a method of preventing a head lice infestation in a human patient at risk which comprises topically administering to the human a glutamate-gated chloride ion channel-binding and GABA receptor-binding formulation as disclosed herein by one dose or multiple doses (preferably a single application) so as to prevent infestation in the patient at risk (i.e., a human likely to be in close contact with an infected individual) by a strain of head lice being either a susceptible strain or a treatment resistant strain or any combination of either or both types of strains within a specific human infestation.

Therefore, the present invention relates to a method of eliminating or preventing an infestation of head lice on a human patient, whether the infecting head lice strain be a susceptible strain or a treatment resistant strain by applying one or multiple doses (applications) of the glutamate-gated chloride ion channel-binding and GABA receptor-binding as disclosed herein to the individual for a length of time sufficient to allow biologically effective contact between the active ingredient and the lice.

The present invention also relates to glutamate-gated chloride ion channel-binding and GABA receptor-binding formulations that are effective against pediculosis via pubic lice (Phthirus pubis) and body lice (Pediculus humanis corporis). The formulations of the present invention may be used ‘as is’ to treat a pubic lice and/or body lice infestation, or may be altered to provide for a formulation with the consistency of a cream rinse or lotion that may be applied to the affected area(s), left on for a period of time as contemplated herein for treatment of head lice, and then rinsed off. Multiple dosing may also occur as contemplated herein for treating head lice with a formulation as disclosed herein.

Another aspect of the present invention relates to the use, in manufacture of a topical medicament for treating or preventing a pubic lice or body lice infestation from a treatment resistant strain in a human patient, of avermectin or ivermectin and spinosyn or spinosad.

Another aspect of the present invention relates to the use, in the manufacture of a medicament for treating or preventing a pubic lice or body lice infestation from a susceptible or treatment resistant strain in a human patient, of an active ingredient component combining at least one compound that selectively binds to glutamate-gated chloride ion channels and at least one compound that selectively binds to GABA receptors.

### BRIEF DESCRIPTION OF THE FIGURES

FIGS. 1A and 1B show the chemical structures of avermectin, form B₁ₐ (1A) and B_{1b} (1B).

FIGS. 2A and 2B show the chemical structure for ivermectin, form B₁ₐ (2A) and B_{1b} (2B).

FIG. 3 shows the chemical structure for spinosyn A and D.

### DETAILED DESCRIPTION OF THE INVENTION

The present invention relates to topical formulations combining compounds that selectively bind to glutamate-gated chloride ion channels with compounds that selectively bind to GABA receptors useful as a pediculicidal agents in the treatment or prevention of head lice infestation, where the infested lice may be either a susceptible or treatment resistant strain of Pediculus humanus capitis.

One embodiment of the present invention relates to topical formulations combining compounds that selectively bind to glutamate-gated chloride ion channels with compounds that selectively bind to GABA receptors useful as pediculicidal agents in the treatment or prevention of an infestation of head lice, where the infested lice may be either a susceptible or treatment resistant strain of Pediculus humanus capitis.

The formulations of the present invention address the emerging problem of treatment resistant head lice strains by allowing use of a single product which is active in eliminating or preventing head lice infestations by two modes of action, whether the infestation represents a susceptible or treatment resistant strain of head lice. Accordingly, a portion of the current invention is exemplified by the use of a glutamate-gated chloride ion channel-binding and GABA receptor-binding formulation in an assay to determine killing ability against permethrin-resistant head lice.

It is well known in the art that similar treatment regimes are presently utilized to treat not only a head lice infestation, but also at least an infestation of pubic lice or body lice. Thus, it will be evident that glutamate-gated chloride ion channel-binding and GABA receptor-binding formulations will be effective in treating not only head lice, but also infestations of the human body of pubic lice and body lice. Pubic lice (Phthirus pubis, e.g., “crabs”) are usually found in the pubic area but may also be found on facial hair, on eyelashes, on eyebrows, in the armpits, on chest hair, and, rarely, on the scalp.

Body lice (Pediculus humanis corporis) live and lay eggs in the seams of clothing. The lice are only present on the body when they feed. Under some conditions these lice may transmit diseases such as typhus, relapsing fever, and trench fever. The formulations of the present invention may be used ‘as is’ to treat a pubic lice and/or body lice infestation. Of course, the core components of the disclosed formulation may be altered to provide for a formulation with the consistency of a cream rinse or lotion that may be applied to the affected area(s), left on for a period of time as contemplated for treatment of head lice, and then rinsed off. Multiple dosing may also occur as contemplated herein for treating head lice with a formulation as disclosed herein.

The present invention also relates to a method of treating or preventing a head lice infestation, whether the infestation be from a strain of head lice that remains susceptible to current OTC and prescription pediculicides (“a susceptible strain”) or from a strain that is resistant to one or more available OTC and/or prescription formulations (“a treatment resistant” or “drug-resistant” strain). Thus, one aspect of the invention relates to a method of treatment or prevention of an infestation of head lice on a human patient utilizing a glutamate-gated chloride ion channel-binding and GABA receptor-binding formulation, whether the infecting head lice strain may be a susceptible strain or a treatment resistant strain.

The method applies one dose or multiple doses (applications) of the topical glutamate-gated chloride ion channel-binding and GABA receptor-binding formulation to the individual for a length of time to allow an effective amount of the active ingredient to contact the infecting lice.

An additional aspect of this portion of the invention relates to a method for prophylaxis or elimination of either susceptible or treatment resistant head lice using the glutamate-gated chloride ion channel-binding and GABA receptor-binding formulation as a shampoo-conditioner, where the shampoo-conditioner is used twice during about a seven day period (for example, day 1 and day 7), left on the scalp for about 1 to about 60 minutes at each application, and then rinsed with warm water. The shampoo-conditioner is formulated to leave the hair in good condition while ridding the scalp of lice. Embodiments according to this aspect of the invention leave the formulation on the scalp for between about 3 and about 30 minutes, for between about 5 and about 20 minutes and for between about 10 and about 15 minutes

Thus, another aspect of the present invention relates to the use, in manufacture of a topical medicament for treating or preventing a head lice, pubic lice or body lice infestation from a treatment resistant strain in a human patient, of an active ingredient component combining at least one compound that selectively binds to glutamate-gated chloride ion channels and at least one compound that selectively binds to GABA receptors. Another aspect of the present invention relates to the use, in the manufacture of a medicament for treating or preventing a pubic lice or body lice infestation from a susceptible or treatment resistant strain in a human patient, of a topical formulation according to the various formulations disclosed herein.

One embodiment of the present invention provides topical formulations containing compounds that selectively bind to glutamate-gated chloride ion channels and compounds that selectively bind to GABA receptors that are macrocyclic lactones, or macrolides. More specifically the topical formulations contain at least one avermectin and at least one spinosyn.

The avermectins may be present at varying concentrations (at a weight/volume percentage) of, for example, from about 0.005% to about 5.0% (w/v) [i.e., a 1% avermectin formulation would include 1 gram (g) avermectin per 100 milliliters (ml) (1 g/100 ml)] of formulation volume. The spinosyns may be present at varying concentrations at a weight/volume percentage) of, for example, from about 0.005% to about 5.0% (w/v) [i.e., a 1% spinosyn formulation would include 1 g spinosyn per 100 ml (1 g/100 ml)] of formulation volume.

Another contemplated range for an avermectin concentration would be from about 0.1% to about 2.0% (w/v) of the formulation, which is further supported in Example 1, where an avermectin in the form of ivermectin is present at a concentration of 0.25%. Also note that our studies have shown that ivermectin concentrations of 0.25%, 0.5% and 1.0% promote effective killing of a permethrin-resistant strain of head lice. Contemplated ranges for a spinosyn concentration would be from about 0.1% to about 2.0% (w/v) of the formulation. In Example 1, a spinosyn, in the form of spinosad, combined with ivermectin has a concentration of 0.25% w/v of the total formulation. However, a spinosad or other spinosyn in the formulation herein may be present at varying concentrations including 0.25%, 0.5% and 1.0% (w/v) of the formulation.

Of course, dosing may be varied either by altering the avermectin or spinosyn concentration, as noted above, or by increasing the amount of topical formulation applied to the scalp of the human patient. It is evident upon review of this specification, that, while an ivermectin and spinosad based formulation is useful to practice the present invention, other known avermectins beside ivermectin and other known spinosyns beside sipnosad are also contemplated and have utility as the active ingredient component for the present invention.

While dosing ranges may vary, it is expected that a single application (dosage) to the scalp of an ivermectin/spinosad containing formulation of the present invention would be in the range from about 1 ml to about 100 ml. The present invention also includes embodiments in which the range is from about 3 ml to about 75 ml.

In another embodiment, at least about 60 ml of the topical formulation is applied to totally saturate the roots and to effectively cover the entire scalp area. It is evident upon review of this specification that the artisan may vary the ivermectin and spinosad concentrations and/or volume of the topical formulation to manipulate the effective amount of ivermectin and spinosad to be delivered to the patient's scalp.

Another aspect of the present invention, as noted above, relates to multiple doses of the topical avermectin and spinosyn or ivermectin and spinosad based formulations of the present invention. Multiple applications will include at least one, two, three or four additional dosages beyond the initial dose, with one or possibly two additional doses being most reasonably contemplated.

The initial dose is most likely in response to an infestation of head lice, while the additional subsequent dose(s) are follow up applications to eradicate lice that hatch following the first dose or lice that have somehow survived the first dosing. Multiple dosing for a therapeutic regime provides an opportunity to deliver greater amounts of the active ingredient spaced over a determined time frame.

One aspect of this portion of the invention relates to a method for prophylaxis or elimination of either susceptible or treatment resistant head lice using the glutamate-gated chloride ion channel-binding and GABA receptor-binding formulations as a shampoo-conditioner. The shampoo-conditioner may be used twice during about a seven day period (day 1 and between about day 5 and day 9), as well as three or four times (with an initial application on day 1 followed for a second, third or fourth application at intervals from about 5 days to about 9 days). At each dosing, the formulation remains in the scalp for from about 1 minute to about 60 minutes or from about 3 minutes to about 30 minutes, then rinsed with warm water. The present invention also includes embodiments wherein the formulation remains in the scalp from about 5 minutes to about 20 minutes, and from about 10 minutes to about 15 minutes.

The shampoo-conditioner is formulated to leave the hair in good condition while ridding the scalp of lice. Therefore, the present invention relates to a method of treating a head lice infestation in a human patient which comprises topically administering to the human a formulation as disclosed herein by one dose or multiple doses; the strain of head lice being either a susceptible strain or a treatment resistant strain.

Furthermore, the present invention relates to a method of preventing a head lice infestation in a human patient at risk which comprises topically administering to the human a formulation as disclosed herein by one dose or multiple doses (preferably a single application) so as to prevent infestation in the patient at risk to infestation by a strain of head lice being either a susceptible strain or a treatment resistant strain. Thus, methods of the present invention are useful for preventing head lice infestation in a human susceptible to such infestation.

Such an at risk individual would be a human coming into close contact with an infested individual, such as a family member or possibly in response to an outbreak within a confined space such as school or the workplace. Either a single or multiple dosing regime will be effective, the course of a prophylactic treatment most likely determined by the severity of the outbreak.

Thus, the present invention provides a formula for prophylaxis and elimination of head lice comprising an effective amount of an active ingredient component combining at least one compound that selectively binds to glutamate-gated chloride ion channels and at least one compound that selectively binds to GABA receptors in a shampoo-conditioner. However, the formula may be delivered in various forms, non-limiting examples being creams, gels, pomades, and the like. The invention also provides a method for prophylaxis and elimination of head lice on human hosts comprising topically massaging said formula in a suitable carrier onto and about the infected area.

For the purposes of this invention, the following non-limiting definitions are used:

The term “active ingredient component”, when directed toward the present invention, refers to a combination of at least one compound that selectively binds to glutamate-gated chloride ion channels and at least one compound that selectively binds to GABA receptors. Examples of “compounds that selectively bind to glutamate-based chloride ion channels” include avermectins, which include, but are not limited to, the synthetic derivative ivermectin, as well as avermectin B₁ₐ, avermectin B_{1b}, doramectin, ivermectin, selamectin, eprinomectin, and the like.

Examples of “compounds that selectively bind to GABA receptors” include spinosyns, which include, but are not limited to, individual spinosyn factors (A, B, C, D, E, F, G, H, J, K, L, M, N, O, P, Q, R, S, T, U, V, W, or Y) and any combinations thereof. Ivermectin may be utilized as a mixture of over 80% 22,23-dihydroavermectin B₁ₐ and less than 20% 22,23-dihydroavermectin B_{1b}. As exemplified herein, ivermectin is a mixture of at least 90% 22,23-dihydroavermectin B₁ₐ and less than 10% 22,23-dihydroavermectin B_{1b}.

The term “spinosad” refers to a combination of the spinosyn factors spinosyn A and spinosyn D, where sipnosyn A comprises approximately 85% and sinposyn D comprises approximately 15% of said spinosad.

The term “avermectin/spinosyn” or “avermectin and spinosyn” refers to a combination of one or more avermectins, including but not limited to avermectin B₁ₐ, avermectin B_{1b}, doramectin, ivermectin, selamectin, and eprinomectin, and one or more spinosyns, including but not limited to individual spinosyn factors (A, B, C, D, E, F, G, H, J, K, L, M, N, O, P, Q, R, S, T, U, V, W, or Y), or any combinations thereof.

The term “ivermectin/spinosad” or “ivermectin and spinosad” refers to a combination of invermectin, including a mixture of 22,23-dihydroavermectin B₁ₐ and B_{1b}, and spinosad, a combination of the spinosyn factors spinosyn A and spinosyn D, where spinosyn A comprises approximately 85% and sinposyn D comprises approximately 15% of said spinosad. When making up the total (w/v) of the formulation, the ivermectin/spinosad combination can have various percent concentration ranges of the individual insecticides ivermectin and spinosad.

The term “solubilizer(s)” refers to water-soluble or water-miscible surface active agent compounds that initially dissolve the active ingredient components to stabilize the active ingredient components in water. By way of example the solubilizers include the surface active agents polysorbate 80 and cetyl acetate.

The term “suspending agent(s)” refers to organic compounds that suspend the pre-dissolved active ingredient components before being dispersed in water. Suspending agents include, without limitation, olive oil, shea butter, coco butter, vegetable oil, and the like.

The term “preservative(s)” refers to compounds that reduce the growth of fungus and other bacterial agents. In one embodiment, preservatives are selected from the compounds in the paraben family.

The term “non-ionic surfactant(s)” refers to compounds which act at the water-air and water-oil interfaces, thereby enhancing wetting ability, emulsion stabilization, foaming, rheology, antistatic, lubricity, and surface conditioning properties of the shampoo-conditioner.

The term “emollient(s)” refers to compounds that soften and makes the skin smooth by preventing skin from losing moisture.

The term “silicone compound” refers to hair conditioning agents that change the texture, feel, and appearance of human hair. In one embodiment, the silicone compound is cyclomethicone. Other conditioning agents may also be used.

The term “susceptible” or “susceptible strain” refers to a strain of head lice which is susceptible to one or more available OTC and/or prescription pediculicides, having active ingredients selected from the group consisting of pyrethrins, permethrins, lindane, and malathion where the active ingredient is at least at the pharmaceutically acceptable treatment concentration allowable for that respective pediculicide(s) and all or effectively all head lice are killed at that concentration. In other words, a susceptible strain is a strain which will be effectively controlled following treatment with a pharmaceutically acceptable concentration for that respective pediculicide.

The term “treatment resistant” or “drug resistant” or any such similar term refers to a strain of head lice which shows resistance to one or more available OTC and/or prescription pediculicides having active ingredients selected from the group including, but not limited to one or more pyrethrins, permethrin, lindane, and malathion, where the active ingredient is at least at the pharmaceutically acceptable treatment concentration recommended or allowed for that respective pediculicide(s) and at least some head lice survive at that concentration. Pyrethrins which are approved for use on humans are extracted from the chrysanthum flower, of which there are six active constituents which are esters of two carboxylic acids (chrysanthemic acid and pyrethric acid) and four cyclopentenoles (pyrethrolone, cinerolone, and jasmolone I and II).

So, it is evident that a treatment resistant strain of lice is a strain that shows a measurable level of resistance to killing by a specific pediculicide treatment regime as compared to a susceptible strain, where all or effectively all of the head lice will be killed. Such a measure may be made by an art-accepted assay, for example, as disclosed herein in Example section 1; or may possibly be determined by other means (e.g., comparative testing in individuals).

Topical formulations according to the present invention are aqueous dispersions of an oil phase containing the active ingredient component. The aqueous dispersions may be prepared as water-in-oil emulsions or oil-in-water emulsions, depending upon the rheological properties desired by the product formulator. Water-in-oil emulsions have a gel-like consistency, while oil-in-water emulsions have a lotion-like consistency. How the disclosed ingredients of the present invention may be formulated as water-in-oil or oil-in-water emulsions is well understood by one of ordinary skill in the formulation of personal care products such as shampoos and hair conditioners.

The present invention is exemplified by, but in no way limited to, a topical ivermectin and spinosad containing formulation as disclosed in Table 1. This final formulation is in the consistency of a shampoo-conditioner and at least comprises an effective amount of ivermectin and spinosad, as well as a solubilising agent(s), water, a suspending agent(s), a surfactant(s), silicone compound(s), and a preservative(s), in any combination and/or concentration which may be contemplated by the artisan upon review of this specification.

### Solubilizer (Water-Soluble or Miscible Surface Active Agent)

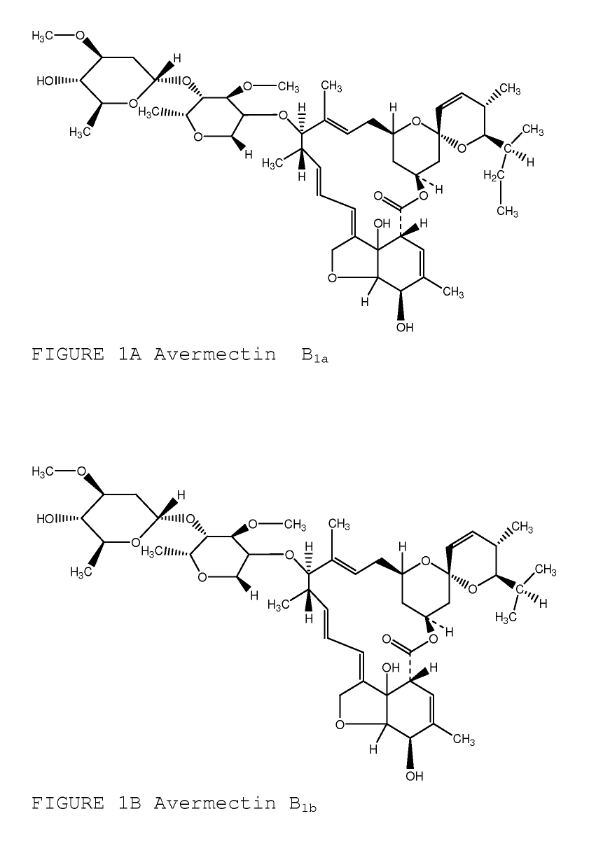

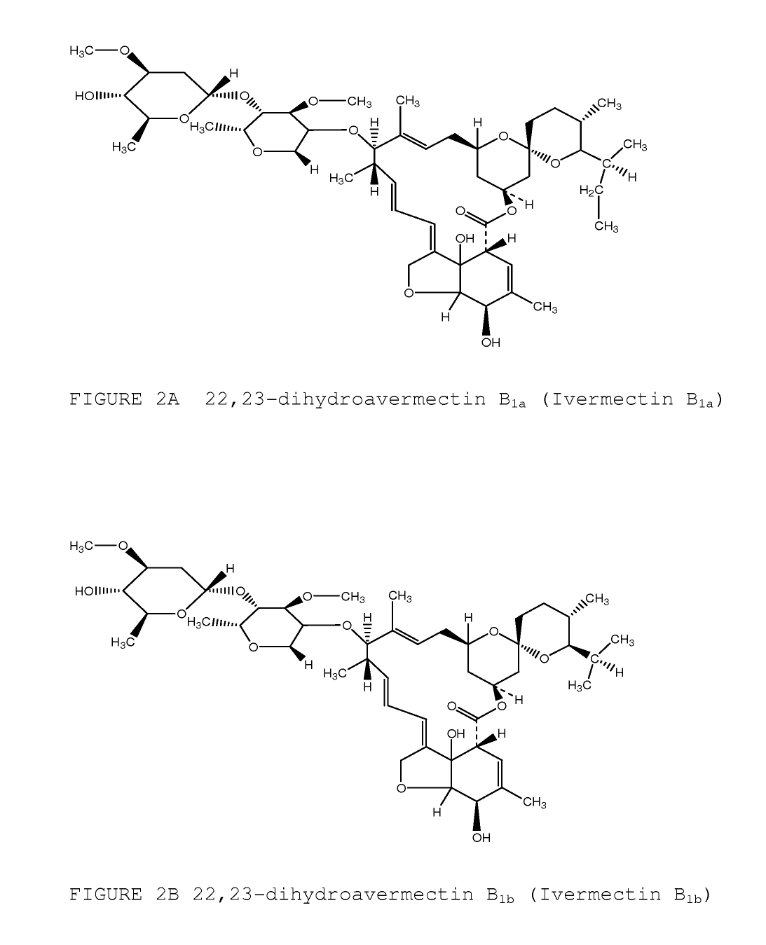

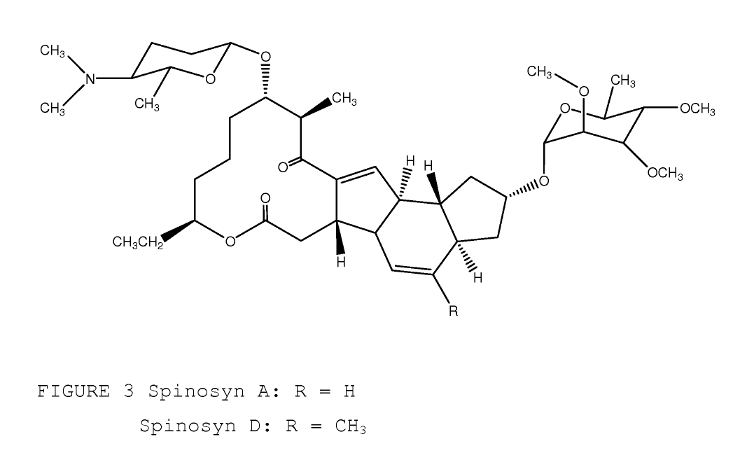

FIGS. 1A and 1B show the chemical formula for avermectin, forms B₁ₐ and B_{1b}. FIGS. 2A and 2B show ivermectin as a mixture comprising about 90% 22,23-dihydroavermectin B₁ₐ and less than about 10% 22,23-dihydroavermectin B_{1b}. FIG. 3 shows spinosyn factors spinosyn A and spinosyn D. The active ingredient component may be delivered to a head lice infested scalp by way of an emulsion based shampoo-conditioner. However the active ingredient component is typically hydrophobic and is poorly solvated by water. Thus, the active ingredient component should preferably be pre-dissolved in suitable surfactants to improve the stability of said active ingredient components in water. Preferably, these surfactants are easily soluble in water or are water miscible, i.e., a water-soluble or water-miscible surface active agent.

In one embodiment the active ingredient component is pre-dissolved in a mixture containing the water miscible organics (water-miscible surface-active agents), e.g. polysorbate 80, cetyl acetate, and acetylated lanolin alcohol. More specifically, an avermectin such as ivermectin and a spinosyn such as spinosad are present in the formula at a level of about 0.05% to 5%, or alternatively from about 0.1% to 2% by weight, or alternatively from about 0.25% to 1% by weight. Polysorbate 80 is present in the formula at a level of about 5% to about 25% by weight, or alternatively from about 10% to 15% by weight (e.g. about 11.25% to 13.5% by weight). Cetyl acetate is present in the formula at a level of about 0.5% to 10% by weight, or alternatively from about 1% to 4% by weight (e.g. about 1.50% to 3.75% by weight). Acetylated lanolin alcohol is present in the formula at a level of about 0.10% to 3% by weight, or alternatively from about 0.5% to about 1% by weight (e.g, 0.15% to 0.75% by weight).

Having these water-miscible surface active agents bound to the surfaces of the active ingredient components will ensure that the active ingredient components will be stable in the aqueous environment of the shampoo-conditioner. In one embodiment of the invention, the active ingredient components may be stabilized by pharmaceutically accepted glycols present in said formula at a level below 30% by weight, such as, for example, below 25%, or below 20% or below 15% or below 10% or below 5%. In another embodiment of the invention said formula does not include any glycols.

### Suspending Agents

Once the active ingredient component is dissolved in a water-soluble or water-miscible surfactant, the active ingredient component is placed in one or more suspending agents. In one embodiment, a combination of fatty oils and fats serve as suspending agents. Olive oil is a triacylglyceride, where three fatty acids are tethered to a glycerol backbone and shea butter is primarily made of palmitic, stearic, oleic, linoleic, and arachidic fatty acids. Although these fatty acids have shown to be adequate home remedies for removal of head lice from the scalp, they do not kill head lice. Both olive oil and shea butter are viscous materials that slow the movement of adult lice to better remove them.

Also note that both fatty acids can act as skin moisturizers. In one embodiment, olive oil is present in the formula at a level of about 20% to 30% by weight, or alternatively from about 25% to 28% by weight (e.g., about 27.5% by weight). Shea butter is present in the formula at a level of about 1% to 5% by weight (e.g. about 2% by weight). Other known suspending agents which may be utilized in the formulations and related methods include, but are not limited to, coconut oil, palm oil, cottonseed oil, vegetable oil, soybean oil, olive oil, peanut oil, corn oil, sunflower oil, safflower oil, jojoba oil, canola oil, shea butter, cocoa butter, milk fat, amaranth oil, apricot oil, argan oil, avocado oil, babassu oil, ben oil, algaroba oil, coriander seed oil, false flax oil, grape seed oil, hemp oil, kapok seed oil, meadowfoam seed oil, okra seed oil, perilla seed oil, poppyseed oil, prune kernel oil, pumpkin seed oil, quinea oil ramtil oil, rice bran oil, camellia oil, thistle oil, wheat germ oil and combinations thereof.

### Non-Ionic Surfactants

The formulation may be further stabilized by one or more non-ionic surfactants. In one embodiment of the invention, a fatty alcohol or a mixture of fatty alcohols serve as surfactants. Apart from additional stabilization of the active ingredient, the non-ionic surfactants have various purposes in the surface chemistry of said formula, where said formula is used in a final product comprising a shampoo-conditioner.

In addition to their surface-active properties, fatty alcohols are emollients that make the skin smoother and act at the water-air and water-oil interfaces, thereby enhancing wetting ability, emulsion stabilization, foaming, rheology, antistatic, lubricity, and surface conditioning properties of the formula. Suitable non-ionic surfactants include, without limitations, oleyl alcohol, lanolin alcohol, sorbitan tristerate, bees wax, erucyl alcohol, ricinolyl alcohol, arachidyl alcohol, capryl alcohol, capric alcohol, behenyl alcohol, lauryl alcohol, myristyl alcohol, cetyl alcohol, stearyl alcohol, isostearyl alcohol, oleyl alcohol, palmitoleyl alcohol, linoleyl alcohol, elaidyl alcohol, elaidolinoleyl alcohol, linolenyl alcohol, elaidolinolenyl alcohol and combinations thereof.

In one embodiment, the fatty alcohols are oleyl alcohol and lanolin alcohol with combined concentration of about 10% to 35%, or alternatively about 18% to about 24% by weight. More specifically, oleyl alcohol is present in the formula at a level of about 5% to 15% by weight (e.g. about 10% by weight). Lanolin alcohol is present in the formula at a level of about 3% to about 15% by weight, or alternatively from about 5% to 10% by weight (e.g. about 8% by weight).

Sorbitan tristearate is available commercially as Glycomul® TS (Lonza, Inc.) or SPAN 65 as sold by Merck Schuchardt OHG. Sorbitan tristearate is a low HLB ester based surfactant and has many uses in the food and cosmetic industries. The chemical structure of sorbitan tristearate is defined by a cyclic five member ether, with hydroxyl groups, and three fatty acid side chains. Sorbitan tristearate is present in the formula at a level of about 0.1% to 3% by weight, e.g. about 0.5% by weight.

### Silicone Compound (Conditioner Agent)

After the active ingredient components have been pre-dissolved and stabilized by surfactants, a silicone compound may be added. In one embodiment, the silicone compound is selected from a group consisting of volatile silicones, of which cyclomethicone is one. Cyclomethicone may act as a conditioner in the shampoo-conditioner. It gives a soft, silky feel to hair and evaporates quickly leaving little residue. Cyclomethicone is present in the formula at a level of about 1% to 5% by weight, e.g. about 3% by weight. Thus conditioners that may be used include but are in no way limited to cyclomethicone, dimethicone, hexamethyldisiloxane, octamethyltrisiloxane, decamethyltetrasiloxane, dodecameth-ylpentasiloxane, polydimethylsiloxanes and combinations thereof.

### Preservatives

In yet another embodiment, the formula of the instant invention further contains a preservative, protecting the formula from fungus by chemicals. In one embodiment, the preservative is chosen from the paraben family. More specifically, the preservative is selected from methylparaben, propylparaben, or a combination thereof. In one embodiment, the concentration of the preservative is about 1% by weight. More specifically, methylparaben is present in the formula at levels of about 0.01% to 2% by weight (e.g. 0.20% by weight). Propylparaben is present in the formula at levels of about 0.01% to 1.0%, or alternatively from about 0.01% to about 0.5% by weight (e.g., 0.05% by weight). Also note that representative preservatives may include, but are not limited to, methylparaben, propylparaben, ethylparaben, butylparaben, isobutylparaben, isopropylparaben, benzylparaben and their sodium salts thereof.

Other suitable preservatives include sodium benzoate, imidazolidinyl ureas such as the GERMALL family of preservatives, including GERMALL PLUS, polyvalent chelating agents such as EDTA and related compounds in various stages of protonation, and citric acid and sodium salts thereof, either alone, or in combinations of sodium salt and free acid. The other suitable preservatives can also be present in the formula in the same concentration range as the parabens, i.e., at levels of about 0.01% to 2.0%, or alternatively from about 0.01% to about 0.5% by weight (e.g., 0.05% by weight). In one embodiment, between about 0.01% to about 0.1% citric acid is used in combination with about 1.0% to about 1.25% sodium citrate.

### Humectants

In another embodiment, the formulation further contains a humectant. Humectants are hygroscopic materials intended to prevent the formulation from drying out during the course of treatment and prior to rinsing from the hair and scalp. The humectant may also function as a moisturizer in shampoo and conditioner formulations. Humectants are usually molecules with several hydrophilic groups, such as hydroxyl, amine, carboxylic acid groups and esters thereof that provide the molecule the ability to form hydrogen bonds with water molecules.

In one embodiment, other components of the formulation serve a dual role as the humectant, such as many of the non-ionic surfactants, including, but not limited to oleyl alcohol, lanolin alcohol, acetylated lanolin alcohol, and the like. In another embodiment, the humectant is chosen from glycerine, glyceryl triacetate, sorbitol, xylitol, maltitol, polydextrose, quillaia, lactic acid, urea, and the like, and mixtures thereof.

### Water

The oil mixture containing the pre-dissolved active ingredient, suspending agents, surfactants, a silicone compound and preservatives is then dispersed in water. In one embodiment, water is deionized. Water acts as a carrier and may be included as warranted for any respective formulation. In the exemplified formulation, water is present in the formula at a level of about 30% to 40% by weight (e.g. 33% by weight).

The addition of the oily mixture with the active ingredient to deionized water results in a colloidal suspension of the active ingredient, where micelles form around the active ingredient and are arranged such that hydrophilic heads of the surfactants are in contact with the solvent water molecules and the hydrophobic tails of the surfactants are in contact with the active ingredient. This formulation is especially suitable delivering the active ingredient in a shampoo-conditioner, which gives the shampoo-conditioner a suitable washout and flow behavior leaving the hair in good condition.

Specific ivermectin and spinosad concentrations are shown in Table 1 and include 0.70% (w/v) of ivermectin and 0.30% (w/v) of spinosad. This concentration range is presented to simply exemplify the invention and in no way limits the effective range that may be utilized by the artisan to practice the claimed invention. As noted above in this Detailed Description of the Invention section, the artisan may choose an appropriate range of ivermectin and spinosad concentrations (weight/volume [w/v]) anywhere in the range from about 0.005% to about 5% of the total (w/v) of the formulation. Of course, the w/v ratio of the formulation may be adjusted to take into account differing amounts of ivermectin and spinosad on a g/l basis.

TABLE 1

| Ingredient | % (by weight) |
|---|---|
| Ivermectin | 0.70 |
| Spinosad | 0.30 |
| Deionized Water | 33 |
| Olive Oil USP | 27.25 |
| Polysorbate 80 (and) | 11.75 |
| Cetyl Acetate (and) | 3.0 |
| Acetylated | .25 |
| Lanolin Alcohol | |
| Oleyl Alcohol | 10 |
| Lanolin Alcohol | 8 |
| Cyclomethicone | 3 |
| Shea Butter | 2 |
| Sorbitan Tristearate | 0.5 |
| Methylparaben | 0.2 |
| Propylparaben | 0.05 |

Adjustments may easily be incorporated with components, known equivalent components, combinations of components, and respective concentrations to provide for alternative formulations for uses disclosed herein.

Thus, the artisan will be aware that the percentage by weight of any component may be adjusted to compensate for the concentration of the active ingredient, the texture or rheology of the topical formulation and whether it is formulated as a water-in-oil or oil-in-water emulsion (e.g., shampoo, cream, gel) and that components may be added at differing concentrations or may be left out of a formulation or substituted with an equivalent component so as to provide for a topical formulation which possesses efficacy against treatment resistant strains of head, pubic or body lice similar to the exemplified topical formulation described herein.

A person of ordinary skill in the art will appreciate that other beneficial agents can be added into a formula of the instant invention. Such beneficial agents include, without limitation, vitamins, hair dyes, nutrients, anti-dandruff agents and the like.

A person of the ordinary skill will have sufficient expertise to properly select the beneficial agent or the combination thereof depending on the specific embodiment of the invention, so that the at least one beneficial agent would not negate the beneficial aspects of the formula.

One of ordinary skill in the art would appreciate that the formulation and method described herein for eliminating treatment resistant lice is not meant to be a limiting disclosure, but only gives a preferred embodiment of the invention.

### EXAMPLE

### Example 1

### Formulation of a Ivermectin/Spinosad Shampoo Conditioner on Permethrin Resistant Head Lice

Having described the invention in general terms, the following specific example is offered for purposes of illustration and only for illustration, and no intention to limit the invention is to be inferred therefrom. A pediculicide containing ivermectin and spinosad as a topical shampoo-conditioner for eliminating treatment resistant lice may be prepared as follows:

The formulation as shown in Table 1 can be made by the following non-limiting example. Initially, the 1% w/v for the formulation includes ivermectin (at 0.70% by dry weight) and spinosad (at 0.30% by dry weight). The active ingredients are weighed, mixed together, and then pre-dissolved in a vessel containing water-miscible surface active agents, hereinafter Phase A, where the surface active agents include 11.75% w/v of polysorbate 80, 3% w/v of cetly acetate, and 0.25% w/v of acetylated lanolin alcohol. Phase A is heated, with mixing, at a constant temperature of 65° C. until the active ingredient is completely dissolved in the surface active agents. Phase A is then poured into vessel containing Phase B, which consists of suspending agents, preservatives, non-ionic surfactants, humectants, and a conditioner agent.

Phase B consists of 27.25% w/v of olive oil, 2% w/v of shea butter, 8% w/v of lanolin alcohol, 3% w/v of cyclomethicone, 0.50% w/v of sorbitan triesterate, 0.20% w/v of methyl paraben and 0.05% w/v of propyl paraben. Phases A and B are heated, with mixing, at a constant temperature of 85° C. until all ingredients are dissolved and/or melted. Concurrently Phase C, consisting of water, is heated at a constant temperature of 85° C. With vigorous mixing Phases A and B are slowly added to Phase C. Mixing is continued at or near room temperature until a uniform, homogenous mixture is formed, which is then subsequently packaged.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A topical formulation for prophylaxis or elimination of susceptible and treatment resistant head lice comprising an aqueous dispersion of an oil phase comprising:
an effective amount of an active ingredient component comprising at least one compound that binds to glutamate-gated chloride ion channels and at least one compound that binds to gamma-amino butyric acid (GABA) receptors, wherein the active ingredient component is dissolved in at least one water-soluble or water-miscible surface active agent with one or more suspending agents and one or more non-ionic surfactants.

2. The topical formulation of claim 1, wherein said glutamate-gated chloride ion channel-binding compounds and said GABA receptor-binding compounds are macrocyclic lactones.

3. The topical formulation of claim 2, wherein at least one compound that binds to glutamate-gated chloride ion channels is an avermectin.

4. The topical formulation of claim 3, wherein said avermectin is selected from the group consisting of ivermectin, doramectin, selamectin, abamectin and combinations thereof.

5. The topical formulation of claim 2, wherein at least one compound that binds to GABA receptors is a spinosyn.

6. The topical formulation of claim 5, wherein said spinosyn is selected from the group consisting of spinosyn factors A, B, C, D, E, F, G, H, J, K, L, M, N, O, P, Q, R, S, T, U, V, W and Y and combinations thereof.

7. The topical formulation of claim 1, wherein said surface active agent is a water-miscible surface active agent selected from the group consisting of polysorbate 80, cetyl acetate and combinations thereof.

8. The topical formulation of claim 1, wherein said suspending agent is selected from the group consisting of olive oil, shea butter and combinations thereof.

9. The topical formulation of claim 1, wherein said non-ionic surfactant is selected from the group consisting of oleyl alcohol, lanolin alcohol, acetylated lanolin alcohol, sorbitan tristearate and combinations thereof.

10. The topical formulation of claim 1, further comprising a humectant.

11. The topical formulation of claim 1, further comprising a preservative.

12. The topical formulation of claim 1, further comprising a conditioner.

13. The topical formulation of claim 1, wherein said aqueous dispersion is an oil-in-water emulsion.

14. The topical formulation of claim 1, wherein said aqueous dispersion is a water-in-oil emulsion.

15. A topical formulation for prophylaxis or elimination of susceptible and treatment resistant head lice comprising an aqueous dispersion of an oil phase comprising:
an effective amount of an active ingredient component comprising at least one avermectin and at least one spinosyn, wherein the active ingredient component is dissolved in at least one water-soluble or water-miscible surface active agent with one or more suspending agents and one or more non-ionic surfactants.

16. The topical formulation of claim 15, wherein said surface active agent is a water-miscible surface active agent selected from the group consisting of polysorbate 80, cetyl acetate and combinations thereof.

17. The topical formulation of claim 15, wherein said suspending agent is selected from the group consisting of olive oil, shea butter and combinations thereof.

18. The topical formulation of claim 15, wherein said non-ionic surfactant is selected from the group consisting of oleyl alcohol, lanolin alcohol, acetylated lanolin alcohol, sorbitan tristearate, and combinations thereof.

19. The topical formulation of claim 15, further comprising a humectant.

20. The topical formulation of claim 15, further comprising a preservative.

21. The topical formulation of claim 15, further comprising a conditioner.

22. The topical formulation of claim 15, wherein said aqueous dispersion is an oil-in-water emulsion.

23. The topical formulation of claim 15, wherein said aqueous dispersion is a water-in-oil emulsion.

24. A topical formulation for prophylaxis or elimination of head lice, which comprises an aqueous dispersion of an oil phase comprising:
a) an effective amount of ivermectin and spinosad at a combined level of about 0.1% to 5% weight/volume;
b) a water-miscible surface active agent;
c) a suspending agent in the combination of olive oil present from 20% to about 35% weight/volume and shea butter at a level up to about 5% weight/volume;
d) a non-ionic surfactant in the combination of oleyl alcohol present from about 5% to 15% weight/volume, lanolin alcohol present from about 5% to 10% weight/volume, and acetylated lanolin alcohol from about 10%-20% weight/volume;
e) sorbitan tristearate up to a level of about 2% weight/volume;
f) a preservative combination of methylparaben up to about 1.0% weight/volume and propylparaben up to about 0.5% weight/volume; and
g) a conditioner which is cyclomethicone up to about 3% weight/volume;
wherein said ivermectin and spinosad are dissolved in said water-miscible surface-active agent with said suspending agent, said non-ionic surfactant, said sorbitan tristearate, said preservative combination and said conditioner.

25. A method of treating a head lice infestation from a susceptible or treatment resistant strain in a human patient which comprises topically administering to the scalp of a human patient a topical formulation, wherein said topical formulation comprises an aqueous dispersion of an oil phase comprising:
an effective amount of an active ingredient component comprising at least one compound that binds to glutamate-gated chloride ion channels and at least one compound that binds to gamma-amino butyric acid (GABA) receptors, wherein the active ingredient component is dissolved in at least one water-soluble or water-miscible surface active agent with one or more suspending agents and one or more non-ionic surfactants.

26. The method of claim 25, wherein said glutamate-gated chloride ion channel-binding compounds and said GABA receptor-binding compounds are macrocyclic lactones.

27. The method of claim 26, wherein at least one compound that binds to glutamate-gated chloride ion channels is an avermectin.

28. The method of claim 26, wherein at least one compound that binds to GABA receptors is a spinosyn.

29. The method of claim 25, wherein the infestation of head lice in the human patient is from a strain of head lice resistant to at least one pediculicide selected from the group consisting of a pyrethrin, permethrin, lindane and malathion.

30. The method of claim 25, wherein the topical formulation is administered to the human patient as a single dose.

31. The method of claim 25, wherein the topical formulation is administered to the human patient in either two doses, three doses or four doses, with an interval of time between any two consecutive doses being from 5 to 9 days.

32. The method of claim 25, wherein the topical formulation is administered to the human patient, and remains on the scalp for a period from about 1 minute to about 60 minutes.

33. A method of treating a head lice infestation from a susceptible or treatment resistant strain in a human patient which comprises topically administering to the scalp of a human patient a topical formulation, wherein said topical formulation comprises an aqueous dispersion of an oil phase comprising:
an effective amount of an active ingredient comprising at least one avermectin and at least one spinosyn, dissolved in at least one water-soluble or water-miscible surface active agent with one or more suspending agents and one or more non-ionic surfactants.

34. The method of claim 33, wherein at least one avermectin is an ivermectin.

35. The method of claim 33, wherein at least one spinosyn is spinosad.

36. The method of claim 33, wherein the infestation of head lice in the human patient is from a strain of head lice resistant to at least one pediculicide selected from the group consisting of a pyrethrin, permethrin, lindane and malathion.

37. The method of claim 33, wherein the topical formulation is administered to the human patient as a single dose.

38. The method of claim 33, wherein the topical formulation is administered to the human patient in either two doses, three doses or four doses, with an interval of time between any two consecutive doses being from 5 to 9 days.

39. The method of claim 33, wherein the topical formulation is administered to the human patient, and remains on the scalp for a period from about 1 minute to about 60 minutes.

40. A method of preventing a susceptible or treatment resistant head lice infestation in a human patient which comprises topically administering to the scalp of a human patient a topical formulation, wherein said topical formulation comprises an aqueous dispersion of an oil phase comprising:
an effective amount of an active ingredient component comprising at least one compound that binds to glutamate-gated chloride ion channels and at least one compound that binds to gamma-amino butyric acid (GABA) receptors, wherein the active ingredient component is dissolved in at least one water-soluble or water-miscible surface active agent with one or more suspending agents and one or more non-ionic surfactants.

41. The method of claim 40, wherein said glutamate-gated chloride ion channel-binding compounds and said GABA receptor-binding compounds are macrocyclic lactones.

42. The method of claim 40, wherein at least one compound that binds to glutamate-gated chloride ion channels is an avermectin.

43. The method of claim 40, wherein at least one compound that binds to GABA receptors is a spinosyn.

44. The method of claim 40, wherein the topical formulation is administered to the human patient as a single dose.

45. The method of claim 40, wherein the topical formulation is administered to the human patient, and remains on the scalp for a period from about 1 minute to about 60 minutes.

46. A method of preventing a susceptible or treatment resistant head lice infestation in a human patient which comprises topically administering to the scalp of a human patient a topical formulation, wherein said topical formulation comprises an aqueous dispersion of an oil phase comprising:
an effective amount of an active ingredient comprising at least one avermectin and at least one spinosyn, dissolved in at least one water-soluble or water-miscible surface active agent with one or more suspending agents and one or more non-ionic surfactants.

47. The method of claim 46, wherein at least one avermectin is an ivermectin.

48. The method of claim 46, wherein at least one spinosyn is spinosad.

49. The method of claim 46, wherein the topical formulation is administered to the human patient as a single dose.

50. The method of claim 46, wherein the topical formulation is administered to the human patient, and remains on the scalp for a period from about 1 minute to about 60 minutes.
